(19) United States
(12) Patent Application Publication
(10) Pub. No.: US 2006/0212015 A1
(43) Pub. Date: Sep. 21, 2006

(54) Feminine product disposal container

(76) Inventor: Adrienne Peele, Adelaide, South (AU)

Correspondence Address: GREG O'BRADOVICH, P.C.;Suite A, 295 Culver Street, Lawrenceville, GA, 30045, US

(21) Appl. No.: 11/082,302

(22) Filed: Mar. 17, 2005

(51) Int. Cl. A61F 13/20

(52) U.S. Cl. 604/385.13; 604/385.02

(57) ABSTRACT

A feminine disposal container. The container is a small box with a snap down lid made with a biodegradable cardboard or another material. Used tampons can be placed inside the container, the lid snapped down, and the container disposed in the trash without mess or odor. In one embodiment, the container can be preformed and fresh tampons can be included in the container. In another embodiment, a conventional tampon applicator tube can be precut with perforations. Once the applicator is used, the applicator can be torn along the perforations and formed into the container. In several embodiments, different container types can be preformed or formed from conventional tampon containers.

### BACKGROUND

I. Field of the Invention

The present invention relates generally to the field feminine products, and more particularly to a container for the disposal of used feminine products.

II. Description of the Related Art.

Currently, women experiencing their menstrual cycle often have difficulty in deciding how to dispose of the spent feminine product. In particular, those women who use tampons have to typically wrap the product in toilet tissue and place the wrapped item in the garbage. Flushing the product is typically not an option as the product has expanded from moisture and can clog and damage plumbing. In addition, tampons are sold with an applicator that is also typically disposed. Currently there exists no pre-existing disposal container for tampons.

### SUMMARY

In general, the present invention features a feminine disposal container. In a typical embodiment, the container is a small box with a snap down lid made with a biodegradable cardboard or another material. Used tampons can be placed inside the container, the lid snapped down, and the container disposed in the trash without mess or odor. In one embodiment, the container can be preformed and fresh tampons can be included in the container. In another embodiment, a conventional tampon applicator tube can be precut with perforations. Once the applicator is used, the applicator can be torn along the perforations and formed into the container. In several embodiments, different container types can be preformed or formed from conventional tampon containers.

In general, in one aspect, the invention features a feminine product disposal apparatus, including a generally flat and planar main body having sides and an upper and lower end and a flap located on the upper end of the body.

In one implementation, the apparatus further includes an adhesive strip having a removable protective covering, the adhesive strip located on the flap.

In another implementation, the apparatus further includes comprising a series of fold points located on the main body and on the flap.

In another implementation, the sides can be compressed thereby deforming the body generally along the fold points, forming an opening on the upper end of the container and a hollow interior cavity into which a used tampon can be placed.

In another implementation, the body is a reconfigured tampon applicator.

In another implementation, the container is single use only.

In another implementation, the body includes a protective lining on an inner hollow cavity formed when the sides are compressed.

In another implementation, the apparatus further includes a disinfectant disposed on the body

In another implementation, the apparatus further includes a fragrance disposed on the body.

In another aspect, the invention features a tampon system, including a tampon located within a tampon applicator, wherein the tampon applicator includes a series of perforations such that the perforations can be at least one of cut and torn such that the applicator can be reformed into a tampon disposal container to receive the tampon has been used, the tampon applicator further including one or more adhesive strips such that the portions of the applicator once at least one of cut and torn can be connected together to form the disposal container.

In another aspect, the invention features a tampon system, including a tampon applicator, a tampon located within the tampon applicator and a tampon disposal container for receiving the tampon after it has been used.

In one implementation, the container is single use only.

In another implementation, the container includes a generally flat and planar main body having sides and an upper and lower end and a flap located on the upper end of the body.

In another implementation, the container includes a generally rectangular body having four sides and a closed bottom, a lid hingeably attached to the body, the lid having one or more hinge points and a lip connected to one end of the lid for snapping down onto one of the walls of the body thereby closing the container.

In another implementation, the container includes an inner protective coating to prevent leakage of fluids when the tampon is placed into the container after use.

In another implementation, the system includes a fragrance disposed on the container.

In another implementation, the system further includes disinfectant disposed on the container.

One advantage of the invention is that used or spent tampons can be disposed of in a clean and sanitary manner.

Another advantage of the invention is that women who use tampons can have a pre-existing apparatus and method of disposing the tampon after it has been used.

Another advantage of the invention is that women who use tampons have security that they can efficiently dispose of a tampon when it is used.

Another advantage of the invention is that a woman using the container can place the used tampon in a container without having to touch the tampon any more than in normal use.

Another advantage of the invention is that the container in which the tampon is packaged can be used to dispose of the tampon.

Another advantage of the invention is that the container in which the tampon is package can be preformed to be converted into a disposal container.

Other objects, advantages and capabilities of the invention will become apparent from the following description taken in conjunction with the accompanying drawings showing the preferred embodiment of the invention.

### BRIEF DESCRIPTION OF THE DRAWINGS

FIG. 1 illustrates a perspective view of an embodiment of a feminine product disposal container in a closed position;

FIG. 2 illustrates a perspective view of an embodiment of a feminine product disposal container in an open position;

FIG. 3 illustrates a perspective view of an alternate embodiment of a feminine product disposal container in an open position;

FIG. 4 illustrates a front view of the embodiment of the feminine disposal container of FIG. 3 in a closed position; and

FIG. 5 illustrates a side view of the embodiment of the feminine disposal container of FIGS. 3 and 4 in a closed position.

### DETAILED DESCRIPTION

In general, the embodiments described herein are formed of suitable materials. In a typical embodiment, the containers described herein are formed of biodegradable material such as the cardboard applicators in which tampons are typically placed in for sale and use. The embodiments herein can also be preformed containers in which new tampons are placed for sale. In other embodiments as described below, typical tampon applicators can be modified to be formed into a container after it has been used as an applicator. In such embodiments, guide lines such as perforations can be provided on the applicator along which the user can cut and reattach in order to form a container for the spent tampon disposal. It is understood that in other embodiments, other materials such as plastic can also be implemented.

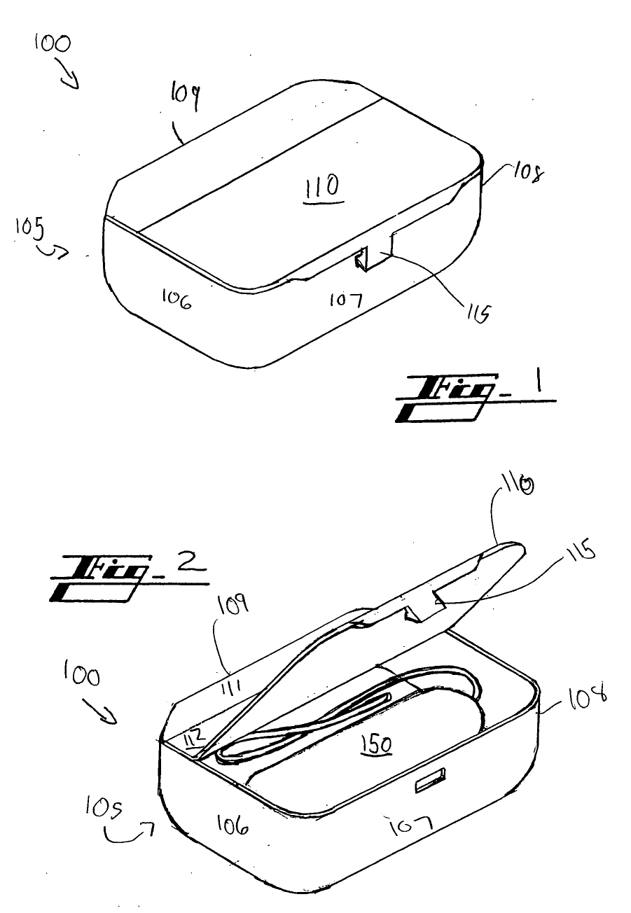

Referring to the drawings wherein like reference numerals designate corresponding parts throughout the several figures, reference is made first to FIG. 1 that illustrates a perspective view of an embodiment of a feminine product disposal container 100 in a closed position. In general, the container 100 includes a main hollow body 105 generally defined by four walls 106, 107, 108, 109. In general, in a typical embodiment, the corners where adjacent walls connect can be rounded giving the container 100 an overall smooth contour and profile. When the container is formed, it is often advantageous for the corners to have a smooth rounded contour. It is understood that in other embodiments, the corners can have other shapes and contours such as typical edges. The container 100 also typically includes a lid 110 hingeably attached to the main body 105. The lid 110 can include a lip 115 located on the end of the lid 110 adjacent wall 107. In general, the lip 115 is available so that the lid 110 can be snapped closed onto the body 105. In another embodiment, the lid 110 can have a main forward lid portion as indicated by lid 110 and a rear lid portion 111. In general, the forward and rear portions 110, 111 allow the container 100 to have an overall increased structural strength.

FIG. 2 illustrates a perspective view of an embodiment of a feminine product disposal container 100 in an open position. As described above, the container 100 generally includes a main hollow body 105 generally defined by four walls 106, 107, 108, 109. The corners can have an overall rounded contour, although other shapes and contours are contemplated. The container 100 also typically includes the lid 110 hingeably attached to the main body 105. The lid 110 can include the lip 115 located on the end of the lid 110 adjacent wall 107. In general, the lip 115 is available so that the lid 110 can be snapped closed onto the body 105. In another embodiment, the lid 110 can have a main forward lid portion as indicated by lid 110 and a rear lid portion 111. In general, the forward and rear portions 110, 111 allow the container 100 to have an overall increased structural strength.

As is further illustrated in the figure, when the lid 110 is opened, the rear portion 111 can further be divided into a third portion 112. It is therefore understood that the lid 110 can have more than one hinge point. In general, since the container 100 can be formed from the actual tampon applicator, those skilled in the art understand that most applicators are thin cardboard. Therefore, when the lid 110 is opened, it is possible that there is more than one hinge point. Since the container is disposable once used, it is appreciated that the structural integrity and repeatability of the opening of the lid 110 is not crucial. Therefore, in the most typical embodiment, the container 100 is for single use only. A tampon 150 is shown placed within the container 100.

Although, in a typical implementation, the container 100 can be formed from a tampon applicator, the container 100 can also be preformed and distributed with tampons as they are normally sold in applicators. Therefore, in another implementation, the container 100 is distributed with tampons as part of a tampon system.

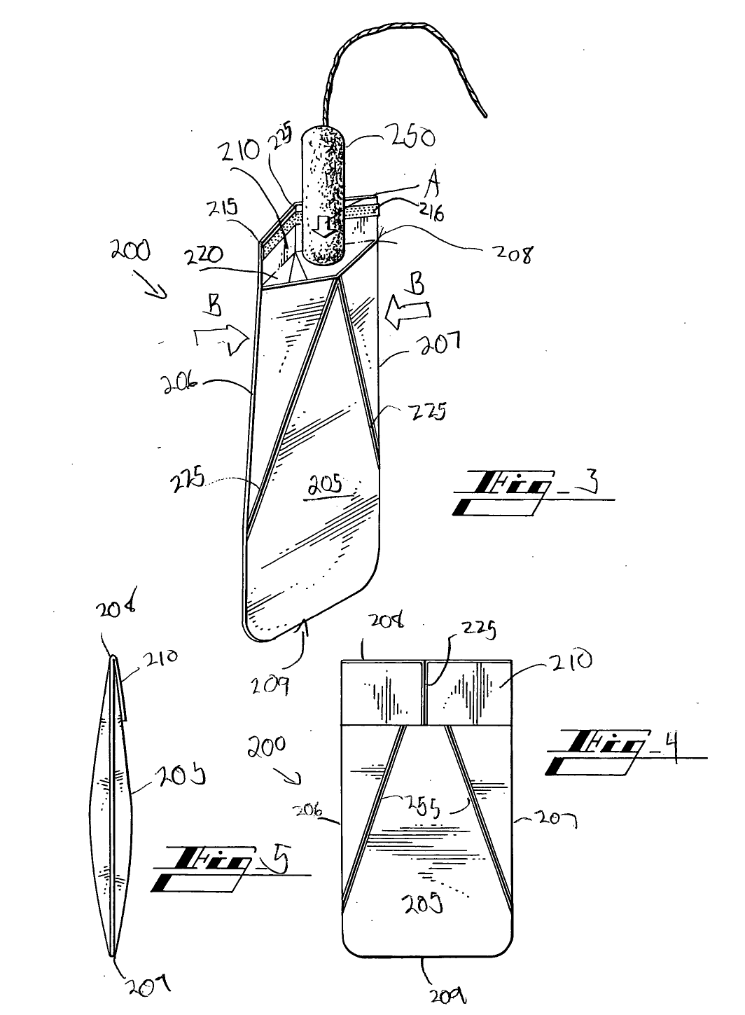

FIG. 3 illustrates a perspective view of an alternate embodiment of a feminine product disposal container 200 in an open position. A tampon 250 is shown as being inserted into the container 200 in the general direction as indicated by arrow A. In general, the container 200 includes an elongated generally planer flat body 205 having sides 206, 207. The body 205 further includes and upper end 208 and a lower end 209. The upper end 208 includes a flap 210 generally including an adhesive strip 215 that can be covered by a protective cover 216 until the adhesive strip 215 is ready for use as discussed further in the description below. It is appreciated that although the body 204 is planar, the body 205 has a finite thickness. It is further appreciated that the body generally include a front piece and a back piece. The back piece being generally longer than the front piece to include the integral flap 210. In general, when the body 205 is in its starting position, it is generally flat. The user can then press the sides 206, 207 generally in the direction as indicated by arrows B. This pressing action generally causes a compression in the body 205 that in turn causes an opening 220 on the upper end of the body 205, and a hollow interior cavity within the body 205. The tampon 250 can then be inserted through the opening 220 into the hollow interior cavity of the body 205.

Since the body 205 is generally used to be compressed for insertion of the tampon 250, the body 205 can typically include a series of fold points 225. In general, it is contemplated that there are a variety of ways that the fold points 225 can be configured. In another embodiment, if the container 200 is configured from a tampon applicator, there may be no need for fold points 225 since the cardboard applicators are made from thin enough cardboard such that compressing the sides 206, 207 to create the opening 220 and a hollow interior cavity can be easily achieved. In general, the fold points 225 as shown in the figure are only for illustrative exemplary purposes. In an embodiment in which the container 200 is manufactured as an additional part of a tampon system in addition to the tampon and its applicator, the cardboard or other material used to make the container 200 can be stiffer. Therefore, the fold points 225 are preformed such that when the container 200 is compressed as described above, the fold points 225 provide creases through which the body 205 can compress. It is appreciated that since the opening 220 can take a variety of shapes such as a diamond shape as shown in the figure for illustrative purposes, the flap 210 can also include one or more fold points 225 so that the container 200 can open generally unimpeded.

It is appreciated that once the tampon 250 is inserted within the hollow interior cavity of the body 205, the protective cover 216 that covers the adhesive strip 215 can be removed, the flap 210 closed over the upper end 208 of the body 205 and sealed. The entire container 200 including the used tampon 250 can then be discarded, typically into a trash receptacle or any other suitable receptacle such as a bio-hazard container, if available.

FIG. 4 illustrates a front view of the embodiment of the feminine disposal container 200 of FIG. 3 in a closed position. The tampon 250 has been inserted into the container 200, the protective cover 216 removed from the adhesive strip, and the flap 210 has been closed and secured to the upper end 208 by using the adhesive strip 215. It is appreciated that since the container 200 includes the used tampon 250, its generally planar and flat profile now includes a bulge. In this configuration, the container 200 is now ready for disposal.

FIG. 5 illustrates a side view of the embodiment of the feminine disposal container 200 of FIGS. 3 and 4 in a closed position. The container 200 includes the used tampon 250 as described above, and therefore has a general bulge as described above.

It is generally appreciated that the container 200 can be formed from a tampon applicator as described above and further below. In another embodiment, the container can be sold and distributed in multiple packs in the flattened state such that multiple containers 200 can be stacked easily on top of one another. In a typical implementation, the containers 200 can be distributed with the tampon and tampon applicators as a self-contained system or kit. The containers can also be packaged and distributed by themselves, without tampons. The embodiments described above can be made commercially available in a variety of ways, including retail and by vending machines.

It is appreciated that in the embodiments described above, the containers can be made from any suitable material. However, it is contemplated that whether or not a cardboard applicator configured to be converted to the container, the container can be a biodegradable material such as cardboard that is generally landfill friendly. In addition, the containers can be coated with any sort of scent and/or disinfectant to help in both odor and disease control. Furthermore, the interior portions of the container can be coated with some sort of leak proof material such as a thin layer of plastic or wax to prevent leakage from the container, typically in case the user must carry the container with used tampon for a period of time. In another embodiment, the containers can optionally include yet another inner bag to receive the used tampon.

It is further appreciated that the embodiments described herein can be formed from pre-existing tampon applicators. Therefore, manufacturers typically do not have to change packaging for multiple tampons. In these embodiments, the applicators include a series of perforations that can be cut or torn such that the applicator can be reformed into one or more of the containers described above. In such embodiments, the applicators generally include adhesive strips with protective covering such that when the applicator is reformed, the protective covers can be removed such that the container can retain its reformed shape. It is understood that it is preferable to keep the adhesive strips away from the user's body. As such, since tampon applicators are generally cylindrical and in two parts in a telescopic arrangement in which one part partially covers the other part, the adhesive strips can be formed inside the interior of the cylinders or on portions of one cylinder that is covered by the other cylinder. Therefore, the adhesive remains isolated from the user in case the adhesive is exposed. When the applicator is reconfigured, the adhesive strips are also exposed therefore allowing the user to efficiently and advantageously reconfigure the applicator in to one or more of the containers described above.

The foregoing is considered as illustrative only of the principles of the invention. Further, various modifications may be made of the invention without departing from the scope thereof and it is desired, therefore, that only such limitations shall be placed thereon as are imposed by the prior art and which are set forth in the appended claims.

What is claimed is:

1. A feminine product disposal apparatus, comprising:
a generally flat and planar main body having sides and an upper and lower end; and
a flap located on the upper end of the body.

2. The apparatus as claimed in claim 1 further comprising an adhesive strip having a removable protective covering, the adhesive strip located on the flap.

3. The apparatus as claimed in claim 1 further comprising a series of fold points located on the main body and on the flap.

4. The apparatus as claimed in claim 3 wherein the sides can be compressed thereby deforming the body generally along the fold points, forming an opening on the upper end of the container and a hollow interior cavity into which a used tampon can be placed.

5. The apparatus as claimed in claim 1 wherein the body is a reconfigured tampon applicator.

6. The apparatus as claimed in claim 1 wherein the container is single use only.

7. The apparatus as claimed in claim 1 wherein the body includes a protective lining on an inner hollow cavity formed when the sides are compressed.

8. The apparatus as claimed in claim 1 further comprising a disinfectant disposed on the body

9. The apparatus as claimed in claim 1 further comprising a fragrance disposed on the body.

10. A tampon system, comprising a tampon located within a tampon applicator, wherein the tampon applicator includes a series of perforations such that the perforations can be at least one of cut and torn such that the applicator can be reformed into a tampon disposal container to receive the tampon has been used, the tampon applicator further including one or more adhesive strips such that the portions of the applicator once at least one of cut and torn can be connected together to form the disposal container.

11. A tampon system, comprising:
a tampon applicator;
a tampon located within the tampon applicator; and
a tampon disposal container for receiving the tampon after it has been used.

12. The system as claimed in claim 11 wherein the container is single use only.

13. The system as claimed in claim 11 wherein the container comprises:
a generally flat and planar main body having sides and an upper and lower end; and
a flap located on the upper end of the body.

14. The system as claimed in claim 11 wherein the container comprises:
a generally rectangular body having four sides and a closed bottom;
a lid hingeably attached to the body, the lid having one or more hinge points; and
a lip connected to one end of the lid for snapping down onto one of the walls of the body thereby closing the container.

15. The system as claimed in claim 1 wherein the container includes an inner protective coating to prevent leakage of fluids when the tampon is placed into the container after use.

16. The system as claimed in claim 15 optionally comprising a fragrance disposed on the container.

17. The system as claimed in claim 16 optionally comprising disinfectant disposed on the container.
